(19) United States
(12) Patent Application Publication
(10) Pub. No.: US 2023/0110385 A1
(43) Pub. Date: Apr. 13, 2023

(54) NON-INVASIVE DETECTION OF SALIVARY AUTOANTIBODIES

(71) Applicants: THE REGENTS OF THE UNIVERSITY OF CALIFORNIA, Oakland, CA (US); SEOUL NATIONAL UNIVERSITY INDUSTRY FOUNDATION, SEOUL (KR); IT-UNIVERSITY OF COPENHAGEN, COPENHAGEN S (DK)

(72) Inventors: Fang Wei, Northridge, CA (US); Sarah Kamounah, Copenhagen S (DE); Anne Marie Lynge Pedersen, Charlottenlund (DE); Yeong Wook Song, Seoul (KR); David Chia, Santa Monica, CA (US); David T. Wong, Beverly Hills, CA (US)

(21) Appl. No.: 17/905,419

(22) PCT Filed: Mar. 3, 2021
(86) PCT No.: PCT/US21/20722

§ 371 Date: Sep. 1, 2022

Related U.S. Application Data
(60) Provisional application No. 62/984,473, filed on Mar. 3, 2020.

(51) Int. Cl. G01N 33/564; G01N 33/543

(52) U.S. Cl. CPC G01N 33/564; G01N 33/5438; G01N 2800/101; G01N 2800/24

(57) ABSTRACT

A system and method for the detection of autoantibodies in saliva is described. In particular, the system is suitable for detecting an autoantibody in a subject, wherein the presence of the autoantibody is indicative of the presence or increased risk of development of an autoimmune disease or disorder.

### CROSS REFERENCE TO RELATED APPLICATIONS

This application claims priority to U.S. Provisional Application No. 62/984,473, filed Mar. 3, 2020, which is hereby incorporated by reference herein in its entirety.

### STATEMENT REGARDING FEDERALLY SPONSORED RESEARCH OR DEVELOPMENT

This invention was made with government support under U01 DE017593 awarded by the National Institutes of Health (NIH). The government has certain rights in the invention.

### BACKGROUND OF THE INVENTION

Sjögren's syndrome (SS) is a systemic autoimmune disease characterized by the presence of lymphocytic infiltration of the exocrine glands and circulating anti-SSA/Ro and anti-SSB/La autoantibodies (Fox et al., 2002, Scand J Rheumatol Suppl; 116:3-13; Brito-Zerón et al., 2016, Nat Rev Dis Primers, 2:16047; Mariette et al., 2018, N Engl J Med, 378:931-9). Exocrine glands, especially the salivary and lacrimal glands, are affected by the disease leading to salivary and lacrimal gland dysfunction with oral and ocular dryness. The mechanism underlying the development of SS is mainly believed to include a gradual inflammation of the glandular tissue induced by abnormal T- and B-cell responses to the autoantigens SSA and SSB (Brito-Zerón et al., 2016, Nat Rev Dis Primers, 2:16047). However, previous findings also indicate that the epithelial cells of the exocrine glands are not merely target of infiltrating immune cells, but they are actively involved in the autoimmune response (Manoussakis et al., 2010, J Autoimmun, 35:219-244, Alunno et al., 2015, Mediators Inflamm, 2437235). It has been shown that the salivary gland epithelial cells express human leukocyte antigen (HLA) class I molecules, adhesion molecules, tumor necrosis factor (TNF) receptor superfamily member 5 (i.e., CD40) and 6 (i.e., FAS receptor) and the FAS ligand, proinflammatory cytokines and chemokines that in various ways are involved in recruitment, homing, activation, differentiation, proliferation and organization of immune cells (Manoussakis et al., 2010, J Autoimmun, 35:219-244, Tzioufas et al., 2012, J Autoimmun, 39: 4-8). Cytokines produced by the infiltrating lymphocytes further contribute to upregulation of the molecules on the salivary gland epithelial cells. Salivary gland epithelial cells have also been shown to activate and mediate differentiation of CD4+T cells resulting in survival of B-cells (Manoussakis et al., 2010, J Autoimmun, 35:219-244, Tzioufas et al., 2012, J Autoimmun, 39: 4-8; Youino et al., 2012, Curr Pharm Biotechnol, 13:2071-7).

Detection of anti-SSA/Ro and -SSB/La autoantibodies in serum and/or focal lymphocytic sialadenitis in labial salivary gland tissue is essential for the clinical diagnosis of SS (Vitali et al., 2002, Ann Rheum Dis, 61:554-88; Shiboski et al., 2017, Arthritis Rheumatol, 69:35-45; Shiboski et al., 2012, Arthritis Care Res (Hoboken), 64(4):475-87-10). The prevalence of serum anti-SSA/Ro and anti-SSB/La is reported to be 50-70% and 25-40%, respectively (Fayyaz et al., 2016, Rheum Dis Clin North Am, 42:419-34). Moreover, patients with anti-SSA/Ro autoantibodies usually also have a more severe clinical manifestation than those who are seronegative. Analytical detection platforms that can effectively and quantitatively detect anti-SSA/Ro and/or anti-SSB/La in saliva have not been reported. The presence of anti-SSA/Ro and/or anti-SSB/La antibodies in saliva could derive from the secretion of circulating serum antibodies. However, it is possible that these antibodies in the saliva originate from a local production by the infiltrating B cells and plasma cells.

Thus, there is a need in the art for non-invasive systems and methods for detection of autoantibodies that are biomarkers of autoimmune diseases. The present invention satisfies this need.

### SUMMARY OF THE INVENTION

In one embodiment, the invention relates to a device for detecting at least one autoantibody in a subject, comprising an array of units on a substrate, each unit comprising an electrode chip including a working electrode, a counter electrode, and a reference electrode; wherein the working electrode of at least one unit is coated with a conducting polymer embedded or functionalized with at least one capture antigen, wherein at least one capture antigen is a target antigen of an autoantibody.

In one embodiment, the target antigen is the 52 kDa SSA subunit (Ro52) or a fragment thereof, the 60 kDa SSA subunit (Ro60) or a fragment thereof, or the SSB La antigen or a fragment thereof.

In one embodiment, the invention relates to a method of detecting at least one autoantibody in a subject comprising: obtaining a saliva sample from the subject; adding a first portion of the sample mixture to an electrode chip on a device for detecting at least one autoantibody in a subject, comprising an array of units on a substrate, each unit comprising an electrode chip including a working electrode, a counter electrode, and a reference electrode; wherein the working electrode of at least one unit is coated with a conducting polymer embedded or functionalized with at least one capture antigen, wherein at least one capture antigen is a target antigen of an autoantibody; contacting the sample with a secondary antibody, wherein the secondary antibody is linked to a detectable moiety for generating a current; and measuring the current in the electrode chip, wherein a change in current is correlated to the presence of at least one autoantibody in the sample.

In one embodiment, the target antigen is the 52 kDa SSA subunit (Ro52) or a fragment thereof, the 60 kDa SSA subunit (Ro60) or a fragment thereof, or the SSB La antigen or a fragment thereof.

In one embodiment, the autoantibody is an anti-SSA/Ro autoantibody or an anti-SSB/La autoantibody.

In one embodiment, the invention relates to a method of diagnosing a subject as having or being at increased risk of an autoimmune disease, the method comprising: obtaining a saliva sample from the subject; adding a first portion of the sample mixture to an electrode chip on a device for detecting at least one autoantibody in a subject, comprising an array of units on a substrate, each unit comprising an electrode chip including a working electrode, a counter electrode, and a reference electrode; wherein the working electrode of at least one unit is coated with a conducting polymer embedded or functionalized with at least one capture antigen, wherein at least one capture antigen is a target antigen of an autoantibody; contacting the sample with a secondary antibody, wherein the secondary antibody is linked to a detectable moiety for generating a current; and measuring the current in the electrode chip, wherein a change in current is correlated to the presence of at least one autoantibody in the sample, wherein the presence of at least one autoantibody is associated with having or being at increased risk of an autoimmune disease.

In one embodiment, the target antigen is the 52 kDa SSA subunit (Ro52) or a fragment thereof, the 60 kDa SSA subunit (Ro60) or a fragment thereof, or the SSB La antigen or a fragment thereof.

In one embodiment, the autoantibody is an anti-SSA/Ro autoantibody or an anti-SSB/La autoantibody.

In one embodiment, the disease or disorder is Sjogren's Syndrome, Sicca syndrome rheumatoid arthritis, multiple sclerosis, type I diabetes, systemic lupus erythematosus (SLE), “antinuclear antibody (ANA)-negative” SLE, neonatal lupus erythematosus, idiopathic inflammatory myopathies (IIM), mixed connective tissue disease (MCTD), or primary biliary cholangitis (PBC).

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a schema of the EFIRM immuno-assay.

FIG. 2 depicts the results from example experiments showing that the EFIRM immuno-assay for the detection of anti-SSA/Ro was optimized using various concentrations of human anti-SSA/Ro to generate an optimal calibration curve. Anti-SSA/Ro was spiked into 10 unstimulated whole saliva samples collected from non-SS, non-SICCA, controls to demonstrate that the target was titratable as well as demonstrating low background in control subjects. The same process was performed for anti-SSB/La.

FIG. 3 depicts the results from example experiments depicting a comparison of measurements of anti-SSA/Ro in serum (titer levels) and whole saliva (-nA) from randomly selected patient samples. Patients with low levels (ID no. 65, 67-72), medium levels (ID no. 15, 41, 52, 91, 93, 100, 101) and high levels (ID no. 5, 19, 21, 24, 40, 59, 63) of serum anti-SSA/Ro antibody.

FIG. 4A through FIG. 4D depict the results from example experiments depicting EFRIM detection of anti-SSA/Ro (FIG. 4A) and anti-SSB/La (FIG. 4B) in saliva of SS and SICCA patients. Scattered plots of EFIRM anti-SSA/Ro ((FIG. 4C) and EFIRM anti-SSB/La ((FIG. 4D) vs serum measurements. Red and black dots are clinically classified SS and SICCA patients respectively (based on ACR criteria).

FIG. 5A through FIG. 5D depicts the results from example experiments depicting the performance of Saliva EFIRM anti-SSA/Ro immuno-assay. FIG. 5A depicts data plots of EFIRM measurements of salivary anti-SSA/Ro in SS, SICCA and control subjects. FIG. 5B depicts ROC: SS vs Controls. FIG. 5C depicts SICCA vs Controls. FIG. 5D depicts SS+SICCA vs Controls.

### DETAILED DESCRIPTION

The present invention relates to assay systems and methods for detecting autoantibodies in a saliva sample of a subject in need thereof.

In one embodiment, the invention provides an EFIRM assay system in which an antigen target for an autoantibody is incorporated as a capture antigen. In one embodiment, the antigen target is a target for an anti-SSA/Ro or anti-SSB/La autoantibody.

In one embodiment, the invention relates to methods of using the assay systems of the present invention to diagnose the presence or an increased risk of development of an autoimmune disease or disorder. In one embodiment, the invention relates to methods of treating a subject identified as having or being at increased risk of developing an autoimmune disease or disorder. In one embodiment, the disease or disorder is associated with an anti-SSA/Ro or anti-SSB/La autoantibody.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein the terms “alteration,” “defect,” “variation,” or “mutation,” refers to a mutation in a gene in a cell that affects the function, activity, expression (transcription or translation) or conformation of the polypeptide that it encodes. Mutations encompassed by the present invention can be any mutation of a gene in a cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing the mRNA (splice site mutation) or cause a shift in the reading frame (frameshift).

The term “amplification” refers to the operation by which the number of copies of a target nucleotide sequence present in a sample is multiplied.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “marker” or “biomarker” is meant to include a parameter which is useful according to this invention for determining the presence and/or severity of a disease or disorder.

The level of a marker or biomarker “significantly” differs from the level of the marker or biomarker in a reference sample if the level of the marker in a sample from the patient differs from the level in a sample from the reference subject by an amount greater than the standard error of the assay employed to assess the marker, and preferably at least 10%, and more preferably 25%, 50%, 75%, or 100%.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a control.

“Differentially decreased expression” or “down regulation” refers to biomarker product levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold lower or less, and any and all whole or partial increments therebetween than a control.

A “disease” is a state of health of an animal wherein the animal cannot maintain homeostasis, and wherein if the disease is not ameliorated then the animal's health continues to deteriorate.

As used herein, an “instructional material” includes a publication, a recording, a diagram, or any other medium of expression which can be used to communicate the usefulness of a component of the invention in a kit for detecting biomarkers disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The “level” of one or more biomarkers means the absolute or relative amount or concentration of the biomarker in the sample.

The term “marker (or biomarker) expression” as used herein, encompasses the transcription, translation, post-translation modification, and phenotypic manifestation of a gene, including all aspects of the transformation of information encoded in a gene into RNA or protein. By way of non-limiting example, marker expression includes transcription into messenger RNA (mRNA) and translation into protein, as well as transcription into types of RNA such as transfer RNA (tRNA) and ribosomal RNA (rRNA) that are not translated into protein.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “providing a prognosis” refers to providing a prediction of the probable course and outcome of a disease or disorder, including prediction of severity, duration, chances of recovery, etc. The methods can also be used to devise a suitable therapeutic plan.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

“Sample” or “biological sample” as used herein means a biological material isolated from an individual. The biological sample may contain any biological material suitable for detecting the desired biomarkers, and may comprise cellular and/or non-cellular material obtained from the individual.

“Standard control value” as used herein refers to a predetermined amount of a particular protein or nucleic acid that is detectable in a sample, such as a saliva sample, either in whole saliva or in saliva supernatant. The standard control value is suitable for the use of a method of the present invention, in order for comparing the amount of a protein or nucleic acid of interest that is present in a saliva sample. An established sample serving as a standard control provides an average amount of the protein or nucleic acid of interest in the saliva that is typical for an average, healthy person of reasonably matched background, e.g., gender, age, ethnicity, and medical history. A standard control value may vary depending on the protein or nucleic acid of interest and the nature of the sample (e.g., whole saliva or supernatant).

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### DESCRIPTION

The present invention relates to methods for autoantibody detection employing an electrical field induced release and measurement (EFIRM) system. In some aspects, autoantibodies are detected from saliva of patients using the developed assay.

While the present invention is described generally for the detection of Anti-SSA/Ro and Anti-SSB/La autoantibodies in a saliva sample, it should be appreciated that any autoantibody that is present in a saliva sample can be detected using the methods of the invention. Non-limiting examples of such detectible autoantibodies include those associated with autoimmune diseases and disorders. It should be appreciated that any number of autoantibodies can be detected using the assay platform, including, without limitation, 1, 2, 3, 4, 5, 6, 7, 8, 9, 10 or more than 10 autoantibodies.

The noninvasive detection of autoantibodies in a subject via the present invention enables clinicians to identify the presence of an autoimmune disease or disorder in a fast, economical and non-invasive manner.

### EFIRM System

As contemplated herein, the present invention includes a method of detecting autoantibodies using a multiplexing electrochemical sensor. In one embodiment, the device utilizes a small sample volume with high accuracy. In addition, multiple autoantibodies can be measured simultaneously on the device with single sample loading. The device may significantly reduce the cost to the health care system, by decreasing the burden of patients returning to clinics and laboratories.

In one embodiment, the electrochemical sensor is an array of electrode chips (GeneFluidics, USA). In some embodiments, each unit of the array has a working electrode, a counter electrode, and a reference electrode. The three electrodes may be constructed of bare gold or other conductive material before the reaction, such that one or more antigens may be immobilized on the working electrode. Electrochemical current can be measured between the working electrode and counter electrode under the potential between the working electrode and the reference electrode. The potential profile can be a constant value, a linear sweep, or a cyclic square wave, for example. An array of plastic wells may be used to separate each three-electrode set, which helps avoid the cross contamination between different sensors. A conducting polymer may also be deposited on the working electrodes as a supporting film, and in some embodiments, as a surface to functionalize the working electrode. As contemplated herein, any conductive polymer may be used, such as polypyrroles, polanilines, polyacetylenes, polyphenylenevinylenes, polythiophenes and the like.

In one embodiment, a cyclic square wave electric field is generated across the electrode within the sample well. In certain embodiments, the square wave electric field is generated to aid in polymerization of one or more capture antigens to the polymer of the sensor. In certain embodiments, the square wave electric field is generated to aid in the hybridization of the capture antigen with the autoantibody to be detected and/or detector probe (e.g., secondary antibody). The positive potential in the csw E-field helps the antigenic molecules accumulate onto the working electrode, while the negative potential removes the weak nonspecific binding, to generate enhanced specificity. Further, the flapping between positive and negative potential across the cyclic square wave also provides superior mixing during incubation, without disruption of the desired specific binding, which accelerates the binding process and results in a faster test or assay time. In one embodiment, a square wave cycle may consist of a longer low voltage period and a shorter high voltage period, to enhance binding partner hybridization within the sample. While there is no limitation to the actual time periods selected, examples include 0.15 to 60 second low voltage periods and 0.1 to 60 second high voltage periods. In a preferred embodiment, each square-wave cycle consists of 1 s at low voltage and 1 s at high voltage. For hybridization, the low voltage may be around −200 mV and the high voltage may be around +500 mV. In some embodiments, the total number of square wave cycles may be between 2-50. In one embodiment, 5 cyclic square-waves are applied for each surface reaction. With the csw E-field, both the polymerization and hybridization are finished on the same chip within minutes. In some embodiments, the total detection time from sample loading is less than 30 minutes. In other embodiments, the total detection time from sample loading is less than 20 minutes. In other embodiments, the total detection time from sample loading is less than 10 minutes. In other embodiments, the total detection time from sample loading is less than 5 minutes. In other embodiments, the total detection time from sample loading is less than 2 minutes. In other embodiments, the total detection time from sample loading is less than 1 minute.

In one embodiment, a multi-channel electrochemical reader (GeneFluidics) controls the electrical field applied onto the array sensors and reports the amperometric current simultaneously. In practice, solutions can be loaded onto the entire area of the three-electrode region including the working, counter, and reference electrodes, which are confined and separated by the array of plastic wells. After each step, the electrochemical sensors can be rinsed with ultrapure water or other washing solution and then dried, such as under pure N₂. In some embodiments, the sensors are single use, disposable sensors. In other embodiment, the sensors are reusable.

As contemplated herein, the assay platform may be organized as any type of affinity binding assay or immunoassay as would be understood by those skilled in the art. In one embodiment, the present invention is based on the affinity between a capture antigen comprising a target antigen of an autoantibody, an autoantibody of interest, and a secondary antibody for recognition of a bound autoantibody, which functions as a detector probe. In another embodiment, the present invention includes a single platform for multiple autoantibody measurements, instead of a single autoantibody.

In one embodiment, the present invention is efficient in that it is simple, rapid and robust. For example, only small sample volumes are needed (e.g., 10 μl) and less than 10 minutes run time are needed. Multiple marker levels may be provided by the device. By providing statistical analysis the user may have an estimate of their risk, and by utilizing available networking systems, the results can be quickly transmitted for review by a clinician for further assessment.

In one embodiment, an antigen for recognition by an autoantibody is coated onto the electrode and serves as a capture antigen. Exemplary autoantibody target antigens that can be used as capture antigens include, but are not limited to, ribonuceloproteins, histidine-tRNA ligase, snRNP core proteins, Type I topoisomerase, histones, nucleoporin 62, Sp100 nuclear antigen, nucleoporin 210 kDa, ganglioside GQ1B, ganglioside G3D, ganglioside GM1, actin cyclic citrullinated peptide, thrombin, phospholipid, IgG, glutamate receptor, glutamate decarboxylase, voltage-gated potassium channel, neuronal nuclear proteins, thyroglobulin, TSH receptor, vinculin, muscle-specific kinase, voltage-gated calcium channel, nicotinic acetylcholine receptor, aquaporin-4, N-methyl-D-aspartate receptor, and collapsing response mediator protein 5. In one embodiment, the antigen is the 52 kDa SSA subunit or a fragment thereof. In one embodiment, the antigen is the 60 kDa SSA subunit or a fragment thereof. In one embodiment, the antigen is the SSB La antigen or a fragment thereof.

The capture antigen is coated onto the bare gold electrode by applying a cyclic square wave electric field. For example, for each cycle during the coating step, the electric field can be set to +350 mV for 1 s and +950 mV for 1 s. In total, coating of the electrode may proceed for 5 cycles, for a total of 10 s, or however long is deemed necessary. Capture antigens used to functionalize the working electrode surface may be constructed according to any protocol known in the art for the generation of peptides.

After antigen coating, the sensor chip can be rinsed and dried for subsequent sample measurement. Samples containing an autoantibody to be detected, such as a cell-culture medium, a blood sample or a saliva sample, can be mixed with a secondary antibody and transferred onto the electrodes. Hybridization of the autoantibody to the capture antigen occurs during incubation for an appropriate amount of time and in appropriate conditions for the autoantibody to bind to the antigen. Following hybridization, any unbound antibodies can be removed by washing.

Next, the antigen-bound antibodies are detected. In one embodiment, a secondary antibody that binds to the autoantibody to be detected is used as a direct or indirect detector molecule. The detector molecules (e.g, secondary antibodies) can be labeled, such as with fluorescein isothiocyanate, Alexa Fluor, HRP, Biotin, or any other label known in the art. In one embodiment, the secondary antibody is labeled with biotin and is then contacted with a streptavidin bound molecule for generating a detectable readout, allowing for indirect detection of the secondary antibody bound to the autoantibody:antigen complex. In one embodiment, the streptavidin bound molecule comprises poly-horseradish peroxidase. For example, in one embodiment horseradish peroxidase in casein-phosphate-buffered saline can be used, and the 3,3′,5,5′-tetramethylbenzidine substrate for horseradish peroxidase can be loaded, and the amperometric signal measured.

In one embodiment, the detector probe comprises a secondary antibody linked to a detectable label which induces a change in current of the sensor, thereby indicating the binding of the secondary antibody, and autoantibody, with the capture antigen. In certain embodiments, the detectable label itself may be sufficient to alter the current of the sensor. In certain embodiments, the detectable label induces the change in current when it comes into contact with an exogenous reactant. For example, the detectable label may react with the reactant to produce a local change sensed by the electrodes of the sensor to produce an amperometric signal. Therefore, in certain embodiments, the reactant is added to the sensor prior, during, or after the application of the sample to the sensor.

In certain embodiments, the detectable label is directly conjugated to the detector probe. In another embodiment, the detectable label is bound to the detector probe via an intermediate tag or label of the probe. For example, in one embodiment, the detector probe comprises a tag, label, or epitope, which can be used to bind to an antibody or other binding compound harboring the detectable label described above.

Examples of detectable labels and reactants to produce a local change in an electrochemical sensor are well known in the art. In one embodiment, the detectable label comprises HRP and the reactant is TMB, which react to generate an amperometric signal. In another embodiment, the detectable label comprises urease, while the reactant comprises urea.

There is no limitation to the concentrations of such probes used, and may be optimized as needed by the user.

Due to the sensitivity of the present invention, very small volumes may be used to perform the desired assays. For example, the biological sample size from the subject may be between 5-100 microliters. In one embodiment, the sample size need only be about 40 microliters. There is no limitation to the actual or final sample size to be tested.

### Methods of Detecting Autoantibodies

The present invention also relates to methods of detecting at least one autoantibody in a saliva sample of a subject. In one embodiment, the method may be performed as an immunoassay assay and includes the steps of obtaining a sample from the subject, applying the sample to an electrode chip coated with a conducting polymer previously embedded or functionalized with a capture antigen comprising a target antigen of an autoantibody to be detected, or a fragment thereof, contacting the sample with a secondary antibody wherein the secondary antibody is linked to a detectable moiety for generating a current, and measuring the current in the electrode chip. The detectable moiety may be measured, or the magnitude of the current in the sample may be measured, to determine the presence or absence of at least one autoantibody in the sample. In certain embodiments, binding of the autoantibody marker to the electrode of the sensor results in an increase in current or negative current. For example, in one embodiment, binding results in a current in the range of about −10 nA to about −1000 nA.

In one embodiment, the detectable moiety for generating a current is horseradish peroxidase (HRP), which generates a current based on a redox cycle in the presence of hydrogen peroxide.

In one embodiment, the present invention provides methods for diagnosing, determining risk or treating a disease or disorder associated with at least one autoantibody in a subject. Accordingly, the present invention features methods for identifying subjects who are at risk of developing autoimmune diseases, including, but not limited to, rheumatoid arthritis/seronegative arthropathies, osteoarthritis, inflammatory bowel disease, systemic lupus erythematosis, iridoeyelitis/uveitistoptic neuritis, idiopathic pulmonary fibrosis, systemic vasculitis/Wegener's gramilornatosis, sarcoidosis, including, but not limited to, rheumatoid arthritis/seronegative arthropathies, osteoarthritis, inflammatory bowel disease, systemic lupus erythematosis, iridoeyelitis/uveitistoptic neuritis, idiopathic pulmonary fibrosis, systemic vasculitis/Wegener's gramilornatosis, sarcoidosis, myocarditis, postmyocardial infarction syndrome, postpericardiotomy syndrome, subacute bacterial endocarditis (SBE), anti-glomerular basement membrane nephritis, interstitial cystitis, lupus nephritis, autoimmune hepatitis, primary biliary cholangitis (PBC), primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus vulgaris, pityriasis lichenoides et varioliformis acuta, Mucha-Habermann disease, psoriasis, systemic scleroderma, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, Coeliac disease, Crohn's disease, microscopic colitis, ulcerative colitis, antiphospholipid syndrome (APS, APLS), aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, Schnitzler syndrome, undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (Anti-NMDA) receptor encephalitis, balo concentric sclerosis, Bickerstaff s encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, pattern II, Oshtoran Syndrome, pediatric autoimmune neuropsychiatric disorder associated with streptococcus (PANDAS), progressive inflammatory neuropathy, restless leg syndrome, stiff person syndrome, sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Ménière's disease, Behçet's disease, eosinophilic granulomatosis with polyangiitis (EGPA), giant cell arteritis, granulomatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumatic, urticarial vasculitis, vasculitis, and primary immune deficiency.

In one embodiment, the disease or disorder is associated with at least one of an Anti-SSA/Ro or Anti-SSB/La autoantibody. Exemplary diseases associated with at least one of an Anti-SSA/Ro or Anti-SSB/La autoantibody include, but are not limited to, Sjogren's Syndrome, Sicca syndrome, rheumatoid arthritis, multiple sclerosis, type I diabetes, systemic lupus erythematosus (SLE), “antinuclear antibody (ANA)-negative” SLE, neonatal lupus erythematosus, idiopathic inflammatory myopathies (IIM), mixed connective tissue disease (MCTD), and primary biliary cholangitis (PBC; previously referred to as primary biliary cirrhosis), including those subjects who are asymptomatic or only exhibit non-specific indicators of the disease or disorder.

In a number of specific autoimmune diseases, such as Sjogren's Syndrome, autoantibodies appear before the disease clinical onset is presented. The methods of the invention are also useful for monitoring subjects undergoing treatments and therapies for an autoimmune disease or disorder associated with at least one autoantibody, and for selecting or modifying therapies and treatments that would be efficacious in subjects having an autoimmune disease or disorder, wherein selection and use of such treatments and therapies slow the progression of one or more autoimmune disease, or prevent their onset.

The invention provides improved diagnosis and prognosis of an autoimmune disease or disorder associated with at least one autoantibody. The risk of developing an autoimmune disease or disorder associated with at least one autoantibody can be assessed by measuring one or more autoantibody as described herein, and comparing the measured values to reference or index values. Subjects identified as having an increased level of at least one of an autoantibody can optionally be selected to receive treatment regimens, such as administration of prophylactic or therapeutic compounds or treatments to prevent, treat or delay the onset of an autoimmune disease or disorder associated with at least one autoantibody.

Identifying a subject before they develop an autoimmune disease or disorder associated with at least one autoantibody enables the selection and initiation of various therapeutic interventions or treatment regimens in order to delay, reduce or prevent the development or severity of the disease or disorder. In certain instances, monitoring the levels of at least one autoantibody also allows for the course of treatment of the disease or disorder to be monitored. For example, a sample can be provided from a subject undergoing treatment regimens or therapeutic interventions (e.g., drug treatments, immunosuppressive therapy, etc.) for an autoimmune disease or disorder. Samples can be obtained from the subject at various time points before, during, or after treatment.

Data concerning the presence or levels of the autoantibodies of the present invention can also be combined or correlated with other data or test results, including but not limited to imaging data, medical history and any relevant family history.

The present invention also provides methods for identifying agents for treating an autoimmune disease or disorder that are appropriate or otherwise customized for a specific subject. In this regard, a test sample from a subject, exposed to a therapeutic agent, drug, or other treatment regimen, can be taken and the level of one or more autoantibody can be determined. The level of the autoantibody can be compared to a sample derived from the subject before and after treatment, or can be compared to samples derived from one or more subjects who have shown improvements in risk factors as a result of such treatment or exposure.

In some embodiments, these methods may utilize a biological sample (such as urine, saliva, blood, serum, amniotic fluid, or tears), for the detection of one or more autoantibody in the sample. In one embodiment, the sample is a saliva sample. Frequently the sample will be a “clinical sample” which is a sample derived from a patient.

In various embodiments, the level of one or more of markers of the invention in the biological sample of the subject is compared with the level of a corresponding biomarker in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

In some embodiments, the invention provides methods of diagnosing, monitoring the progression of, or treating an autoimmune disease or disorder associated with at least one of an anti-SSA/Ro or anti-SSB/La autoantibody in a subject by assessing the level of one or more of an anti-SSA/Ro or anti-SSB/La autoantibody in a biological sample of the subject.

In various embodiments, the subject is a human subject, and may be of any race, sex and age.

Information obtained from the methods of the invention described herein can be used alone, or in combination with other information (e.g., disease status, disease history, vital signs, blood chemistry, etc.) from the subject or from the biological sample obtained from the subject.

In some embodiments, the level of one or more autoantibody is determined to be increased when the level of the autoantibody detected in a biological sample of a subject is increased by at least 10%, by at least 20%, by at least 30%, by at least 40%, by at least 50%, by at least 60%, by at least 70%, by at least 80%, by at least 90%, or by at least 100%, when compared to with a comparator control.

In one embodiment, a biological sample from a subject is assessed for the level of one or more of an anti-SSA/Ro or anti-SSB/La autoantibody. In some embodiments, the level of one or more of an anti-SSA/Ro or anti-SSB/La autoantibody of the invention is determined to be increased when the level of one or more of an anti-SSA/Ro or anti-SSB/La autoantibody detected in a biological sample of a subject is increased by at least 10%, by at least 20%, by at least 30%, by at least 40%, by at least 50%, by at least 60%, by at least 70%, by at least 80%, by at least 90%, or by at least 100%, when compared to with a comparator control.

Methods of Treatment

The present invention also provides a method of treating or preventing an autoimmune disease or disorder, or reducing at least one symptom associated with an autoimmune disease or disorder in a subject. In one embodiment, the method comprises administering an effective amount of a therapeutic composition to, or performing a therapeutic procedure on, a subject identified by the methods of the invention as having or being at increased risk of developing an autoimmune disease or disorder through detection of an autoantibody in a biological sample of the subject.

In one embodiment, the therapeutic composition comprises at least one therapeutic agent to treat the patient's disease or disorder. In one embodiment, the therapeutic composition comprises at least one therapeutic agent to reducing at least one symptom associated with the patient's disease or disorder.

Exemplary therapeutic agents that can be administered to subjects identified as having or at increased risk of developing an autoimmune disease or disorder include, but are not limited to, immunosuppressant drugs including, but not limited to, corticosteroids (e.g., prednisone, budesonide, and prednisolone), tofacitinib, calcineurin inhibitors (e.g., tacrolimus and cyclosporine), antiproliferative agents (e.g., mycophenolate mofetil, mycophenolate sodium, leflunomide and azathioprine), mTOR inhibitors (e.g., sirolimus and everolimus), biologics (e.g., abatacept, adalimumab, anakinra, certolizumab, etanercept, golimumab, infliximab, ixekizumab, natalizumab, secukinumab, tacilizumab, ustekinumab, and vedolizumab) and monoclonal antibodies (e.g., basiliximab, daclizumab, and muromonab), hydroxychloroquine, methotrexate, cyclosporine, lifitegrast, nonsteroidal anti-inflammatory drugs, pilocarpine, and cevimeline.

Therapeutic compositions can be administered to a subject in need in a wide variety of ways. In various embodiments, the therapeutic composition of the invention is administered orally, intraoperatively, intravenously, intravascularly, intramuscularly, subcutaneously, intracerebrally, intraperitoneally, by soft tissue injection, by surgical placement, by arthroscopic placement, or by percutaneous insertion, e.g., direct injection, cannulation or catheterization. Any administration may be a single administration of a therapeutic composition or multiple administrations. Administrations may be to single site or to more than one site in the subject being treated. Multiple administrations may occur essentially at the same time or separated in time.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are preferably administered by i.v. injection.

The therapeutic composition can be incorporated into any formulation known in the art. For example, the therapeutic composition may be incorporated into formulations suitable for oral, parenteral, intravenous, subcutaneous, percutaneous, topical, buccal, or another route of administration. Suitable compositions include, but are not limited to, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

In the method of treatment, the administration of the composition of the invention may be for either “prophylactic” or “therapeutic” purpose. When provided prophylactically, the composition of the present invention is provided in advance of any sign or symptom, although in particular embodiments the invention is provided following the onset of at least one sign or symptom to prevent further signs or symptoms from developing or to prevent present signs or symptoms from becoming more severe. The prophylactic administration of the composition serves to prevent or ameliorate subsequent signs or symptoms. When provided therapeutically, the pharmaceutical composition is provided at or after the onset of at least one sign or symptom. Thus, the present invention may be provided either prior to the anticipated exposure to a disease-causing agent or disease state or after the initiation of the disease or disorder.

Kits

The present invention further includes an assay kit containing the electrochemical sensor array and instructions for the set-up, performance, monitoring, and interpretation of the assays of the present invention. Optionally, the kit may include reagents for the detection of one or more autoantibody. The kit may also optionally include the sensor reader.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Example 1: Detection of Salivary Anti-SSA/Ro and Anti-SSB/La Autoantibodies Utilizing the EFIRM Platform

As salivary glands are etiological organs in the pathogenesis of Sjögren's Syndrome and anti-SSA/Ro and anti-SSB/La are pathopneumonic autoantibodies for clinical classification for the chronic autoimmune condition, it is important to develop analytical and clinical platforms for detection of these SS autoantibodies in saliva, the fluid that drains the organ that is etiological for disease and its progression. Commercially available bead-based (Luminex) and ELISA-based assays are non-informative for saliva detection, including serum bead-based assays used by CLIA-reference labs. This paper addressed the analytical and clinical abilities to detection the pathopneumonic auto-antibodies, anti-SSA/Ro and anti-SSB/La in saliva of Sjögren's Syndrome patients.

The inability to use commercially available anti-SSA/Ro and anti-SSB/La immunoassays for saliva detection, including clinical labs running serum anti-SSA/Ro and anti-SSB/La assays, necessitate efforts to develop alternative platform for saliva anti-SSA/Ro and anti-SSB/La detection. Electric Field-Induced Released and Measurement (EFIRM) is an electrochemical platform that was developed for detection of salivary omics targets that has demonstrated great utilities for circulating tumor DNA (ctDNA) for liquid biopsy applications. While the design of the EFIRM immuno-assays for anti-SSA/Ro and anti-SSB/La detection is immunoassay-based in nature, it does not involve the use of micro-beads in Luminex assays which can cause non-specificities and the need for sample drying in ELISA process which can also result in non-specific activities.

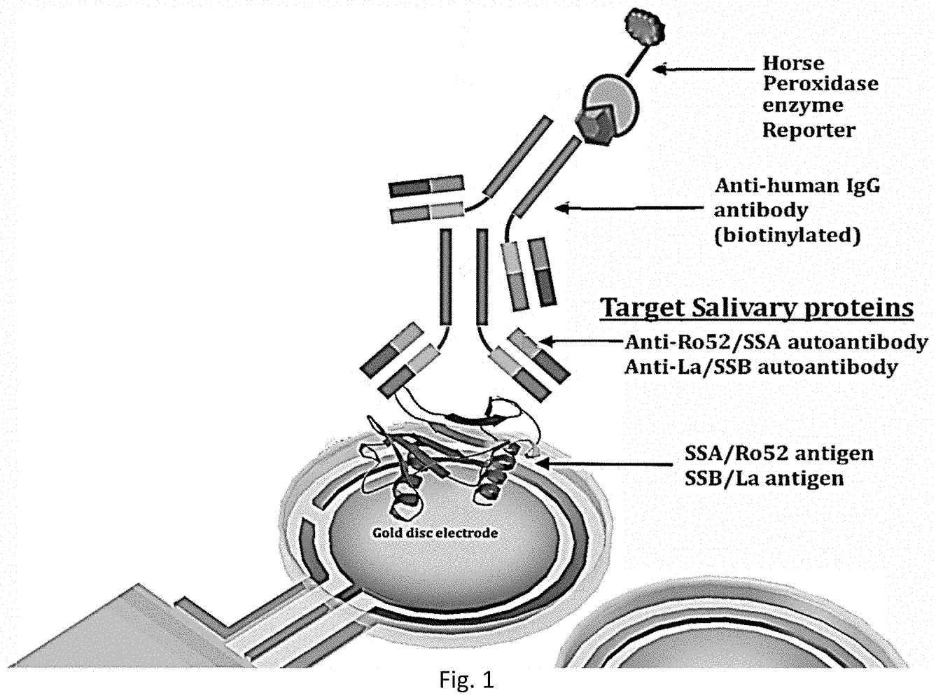

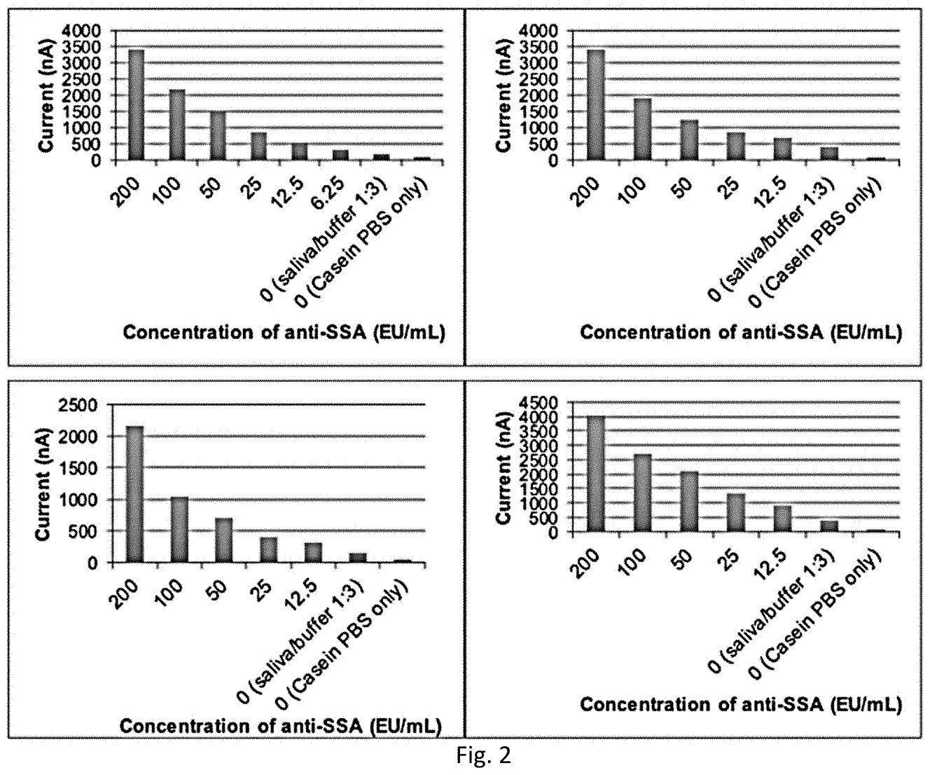

The successful development of EFIRM assays for salivary detection of anti-SSA/Ro and anti-SSB/La was supported by the low background in non-SS, non-SICCA control subjects, titratable spiked in anti-SSA/Ro and anti-SSB/La in control saliva, and significant correlation/concordance of serum to salvia anti-SSA/Ro and anti-SSB/La in SS subjects (FIG. 1 and FIG. 2).

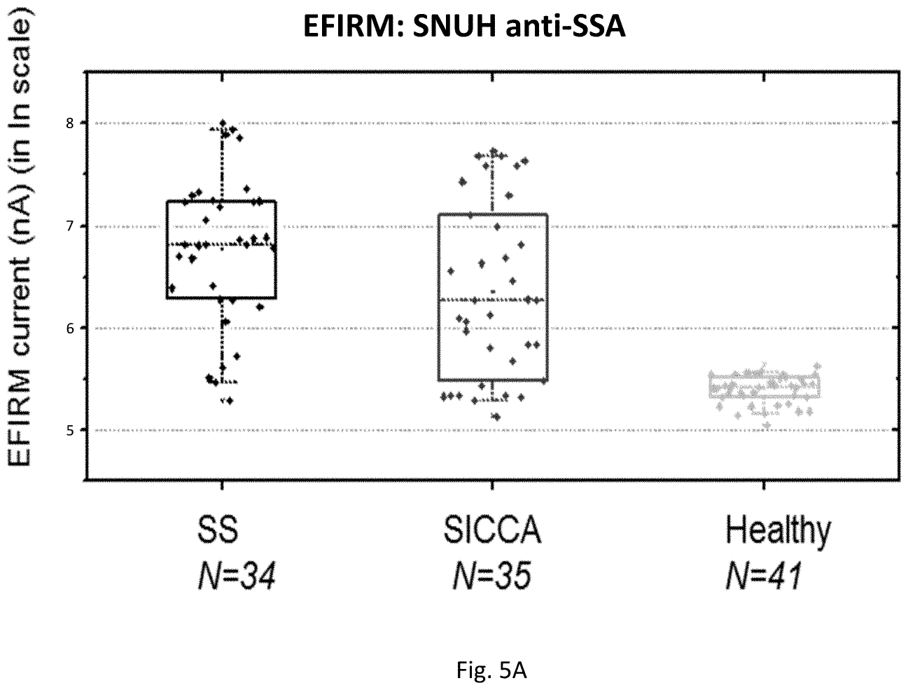

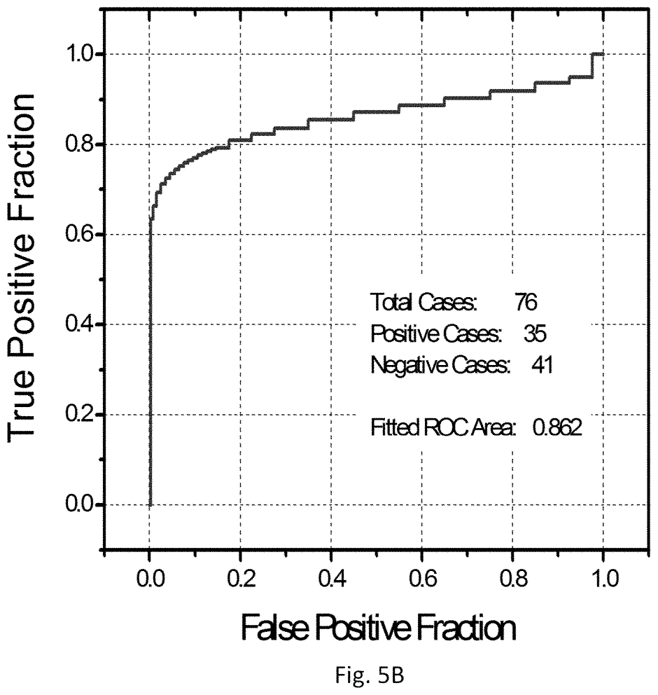

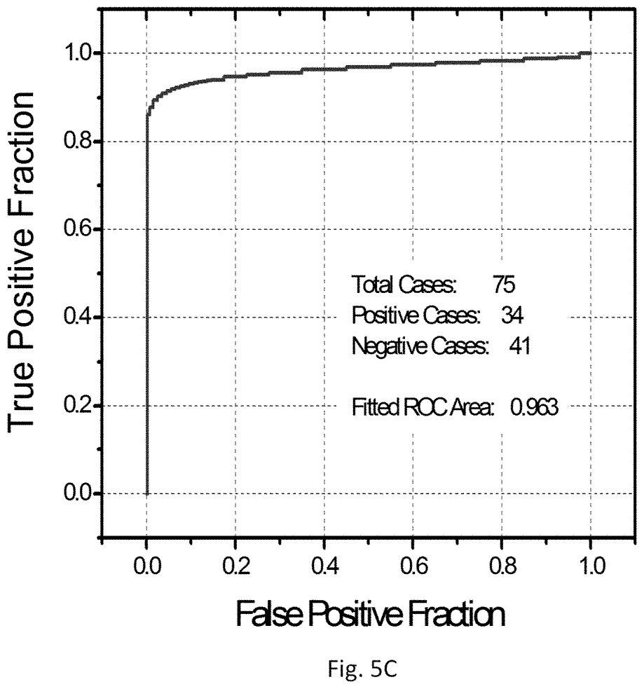

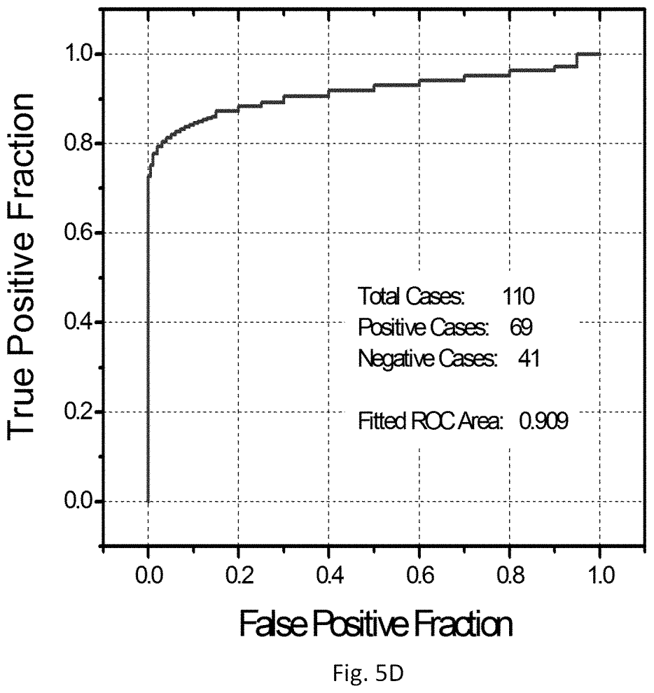

The development of the EFIRM salivary anti-SSA/Ro and anti-SSB/La immunoassays permitted for the first time the ability to examine these pathopneumonic SS autoantibodies in paired saliva and serum of SS and SICCA patients. Using a cohort of SS (33) and SICCA (36) patients, a set of unique and previously unnoticed findings based on saliva determination of anti-SSA/Ro and anti-SSB/La emerged (FIG. 5). SS subjects that are positive for serum anti-SSA/Ro and anti-SSB/La also have detectable anti-SSA/Ro and anti-SSB/La in saliva. SS patients that are serum negative for anti-SSA/Ro (15%) and anti-SSB/La (33%) were all saliva positive for anti-SSA/Ro (100%) and anti-SSB/La (100%), measured by EFIRM. All SS patients (100%) were anti-SSA/Ro and anti-SSB/La positive in saliva. More surprisingly and importantly is the finding that SICCA subjects, by definition serum negative for anti-SSA/Ro and anti-SSB/La, are all positive for these two autoantibodies in saliva (100%).

TABLE 1
Serum and Saliva measurements of anti-SSA/Ro and anti-SSB/La
in Healthy (non-SS, non-SICCA), SS and SICCA Subjects.

| | | Healthy | | |
|---|---|---|---|---|
| | | Non-SICCA, | | |
| | | Non-SS | SICCA | SS |
| Serum | Anti-SSA/Ro (Total Ig) | − | − | + |
| | | | | (85%) |
| | Anti-SSB/La (Total Ig) | − | − | + |
| | | | | (67%) |
| Saliva | Anti-SSA/Ro (Total Ig) | − | + | + |
| | | | (100%) | (100%) |
| | Anti-SSB/La (Total Ig) | ND | + | + |
| | | | (100%) | (100%) |

Inclusion of a non-SS, non-SICCA control group yielded data that support the clinical utilities of the saliva EFIRM immuno-assay for anti-SSA/Ro and anti-SSB/La for SS and/or SICCA risk assessment/screening. The AUC for the ROC evaluations are SS vs Control (0.963); SICCA vs Controls (0.862) and [SS+SICCA] (0.909) (Table 1). The data from this study beholds the promise that a single saliva EFIRM immuno-assay for anti-SSA/Ro or anti-SSB/La can detect all SS and SICCA patients. This can fulfill the unmet clinical need for non-invasive biomarkers for SS detection. More importantly it can fulfill the unmet clinical need for non-invasive biomarker for SICCA early detection, an ability that current does not exist but will be of importance as it can present therapeutic interventions and opportunities for drug development to intervene disease progression and the onset of Sjögren's Syndrome where 5% of SS patients will succumb to B cell lymphoma amongst other quality of life complications.

Of note is that the EFIRM immunoassay for anti-SSA/Ro and anti-SSB/La is plate-based, direct detection assay with no target extraction or procession necessary, requiring only 50 μl of saliva sample.

The detection of anti-SSA/Ro and anti-SSB/La in saliva of SICCA patients suggests that SS originated from pathogenic insults in the salivary glands, releasing of SSA and SSB antigens from damaged glandular epithelial cells and production of anti-SSA/Ro and anti-SSB/La by local plasmocytes. The restriction of saliva anti-SSA/Ro and anti-SSB/La in SICCA patients suggests that SICCA patients are early manifestations of the disease. It is conceivable that upon progression and basement membrane damage which will lead to serum leakage of anti-SSA/Ro and anti-SSB/La, permitting current clinical classification of SS patients. It will be of importance to have the opportunity to monitor longitudinal cohorts of SICCA patients to determine if saliva level of anti-SSA/Ro and anti-SSB/La can predict/forecast Sjögren's disease progression. These capabilities will also permit therapeutic development for intervening disease progression and/or regression.

The mechanism for local production of these salivary autoantibodies is currently unclear. Without being bound by theory, it was hypothesized that salivary gland epithelial cells are not silent bystanders, but actively participate in the pathogenesis by means of acting as antigen-presenting cells presenting SSA/Ro (Brito-Zerón et al., 2016, Nat Rev Dis Primers, 2:16047). This could explain the presence of salivary anti-SSA/Ro in seronegative patients. In this study, a non-specific IgG detector antibody was used, which could suggest that the high levels of salivary anti-SSA/Ro and anti-SSB/La antibodies in serum-negative patients is by virtue of the fact that other isotypes of these autoantibodies were detected in saliva. A next step would therefore be to explore the Ig isotype of salivary anti-SSB/Ro and -SSB/La antibodies.

It should be noted that anti-SSA autoantibodies react against two different SSA-antigens; Ro-52 (52 kDa protein) and Ro-60 (60 kDa protein). In this study the 52 kDa SSA subunit was used as antigen target to capture salivary anti-SSA/Ro. The 60 kDa-target-antigen for anti-SSA/Ro autoantibodies is an RNA-complex with small cytoplasmic RNA (hY-RNA) (Yoshimi et al., 2012, Clin Dev Immunol, 606195). On the other hand, Ro-52 is an interferon (IFN)-induced protein of the tripartite motif family (TRIM) that initially was described as a part of the SSA/Ro ribonucleoprotein (RNP) complex, which is now considered a separate antigen, that can exist both with or without the presence of anti-Ro60 (Yoshimi et al., 2012, Clin Dev Immunol, 606195; Infantino et al., 2015, Arthritis Res Ther, 17:365). The genes encoding Ro52 and Ro60 are located on two different chromosomes, 11 and 19 respectively, and also localized in different cell compartments and associated with different clinical phenotypes (Yoshimi et al., 2012, Clin Dev Immunol, 606195). The 60 kDA SSA subunit can also be used in the EFIRM system as an antigen target to capture salivary anti-SSA/Ro.

This study demonstrates the detection of salivary anti-SSA/Ro and anti-SSB/La in patients with SS by means of the EFIRM platform, suggesting that this analytic platform could contribute to a non-invasive detection of anti-SSA/Ro and anti-SSB/La antibodies utilizing saliva as a SS/SICCA detection risk assessment/screening tool.

The materials and methods used in the experiments are now described.

Study Design and Patients

This cross-sectional study included 69 patients referred for a diagnostic work-up for pSS, of whom 33 patients fulfilled the American College of Rheumatology (ACR) Classification Criteria for Sjögren's syndrome (Shiboski et al., 2012, Arthritis Care Res (Hoboken), 64(4):475-87-10) and classified as pSS patients. The remaining 36 patients with SICCA symptoms did not fulfil the criteria and were classified as non-pSS, SICCA patients. Case definition requires at least 2 out of the following 3:1) Positive serum anti-SSA and/or anti-SSB or [positive rheumatoid factor and ANA≥1:320]; 2) Ocular staining score≥3; 3) Presence of focal lymphocytic sialadenitis with focus score≥1 focus/4 mm2 in labial salivary gland biopsies (Shiboski et al., 2012, Arthritis Care Res (Hoboken), 64(4):475-87-10).

Unstimulated whole saliva samples were collected from all patients for 15 minutes, following the procedure previously described in details (Pedersen et al., 1999, Oral Dis, 5:128-38), kept on ice and centrifuged immediately after collection at 2,600 g for 15 min at 4° C. After having obtained supernatant, 1 μl aprotinin (stock 10 mg/ml), 3 μl Na3OV4 (stock 400 mM) and 10 μl (stock 10 mg/ml) phenylmethylsulfonyl fluoride (PMSF) were added, the tube gently inverted and then divided into 5 aliquots of 200 μl and stored at −80° C. until analysis. Upon analysis the saliva samples were thawed, vortexed for 10 seconds and diluted in phosphate buffered saline (PBS, pH 7.4). All investigators were blinded during the analysis of the saliva samples with regard to the diagnosis of the patients. In all patients, the presence and levels of IgG class antibodies to SSA/Ro and SSB/La in serum were measured by enzyme-linked immunosorbent assays (ZEUS ELISA SSA (Ro) and SSB (La) Test System®, no. 2Z2811G/SM2Z2811G and 2Z2821G/SM2Z2821G, respectively) at the time of diagnostic work-up for pSS.

For the development of immunoassays utilizing the EFIRM technology platform, unstimulated whole samples were collected from 10 healthy subjects with no history of autoimmune disease or intake of medication.

TABLE 2
Demographic characteristics of the patients with pSS and the patients
with SICCA but non-pSS, and the out-come of the diagnostic work-up
according to the ACR classification criteria. Values are given
in mean and SD and in numbers of patients (%).

| | | Patients with | | |
|---|---|---|---|---|
| | Patients with | non-pSS | | |
| | pSS | SICCA | | |
| | (n = 33) | (n = 36) | | P-value |
| Age (yrs.) | 54.2 ± 10.9 | 56.8 ± 14.7 | | NS |
| Female (%) | 33 (100%) | 32 | (89%) | 0.03 |
| Positive serum anti-SSA/Ro | 28 (84.8%) | 1 | (2.8%) | 0.000001 |
| antibody | | | | |
| Serum anti-SSA/Ro antibody | 3.9 ± 4.1 | 0.2 ± 0.4 | | 0.000004 |
| titer (Index Value/OD ratio) | | | | |
| Positive serum anti-SSB/La | 21 (63.6%) | 1 | (2.8%) | 0.000001 |
| antibody | | | | |
| Serum anti-SSB/La antibody | 2.7 ± 2.7 | 0.2 ± 0.6 | | 0.000001 |
| titer (Index Value/OD ratio) | | | | |
| Positive rheumatoid factor (RF) | 19 (57.5%) | 6 | (16.6%) | 0.00001 |
| Positive antinuclear antibody | 29 (87.8%) | 10 | (27.7%) | 0.00001 |
| (ANA) | | | | |
| Labial salivary gland focus | 24 (72.7%) | 0 | (0%) | 0.000001 |
| score ≥1 | | | | |
| Ocular staining score ≥3 | 28 (84.8%) | 6 | (16.6%) | 0.00001 |

The EFIRM (Electric Field-Induced Release and Measurement) Assay Platform

All experimental work for electro-polymerization and electrochemical readout was performed on a custom developed 96-channel electrochemical reader (EZLife Bio, Guangzhou, China). The device consists of a high-throughput electrochemical potentiostat system that is able to apply a fixed voltage and perform electrochemical readout on 96-channels simultaneously (ACEA Biosciences, Inc.). It is connected to the electrode through a pogo-pin system that the electrochemical plate is pressed against. The pogo-pin layout is arranged so as to correspond to the working and counter electrodes on the electrode plate. The pogo pin structure makes it possible to rapidly connect and remove the 96-well plate electrodes with the electrochemical reader and a computer. A USB cable is used to connect the potentiostat control device with a PC computer for control and measurement with a custom-developed electrochemical control software.

Capture Antigen Immobilization

Initially, a mixture of 2.5 μg/mL recombinant SSA/Ro52 or SSB/La antigen (A12700 and A12800; Surmodics Inc, USA), Pyrrole (W338605; Sigma Aldrich, USA) and 3 mM potassium chloride (KCl) was diluted in UltraPure® water. The mixture was vortexed and 30 μl was loaded onto each electrode on the 96-well gold electrode plate (ACEA Biosciences, Inc.). To immobilize the antigen to the surface of the electrode surface, a cyclic square wave electrode field for 5 cycles of is at 350 mV and is of 950 mV was applied (10 s total). After the electrochemical polymerization, each electrode was washed for 3 cycles in a buffer of 1×phosphate buffered saline (PBS, Affymetrix, USA) and 0.05% Tween 20 (BioRad, USA).

Standard Curve

A standard curve was generated using anti-human anti-SSA/Ro52 antibody (LS-C17716; Lifespan Biosciences™, USA) spiked in a PBS solution of 1% w/v purified casein, pH 7.4 (Blocker Casein solution, Thermo Fisher Scientific™, USA). For the anti-SSA/Ro52 assay, the whole saliva samples were diluted in a Blocker Casein solution at a volume ratio of 1:64. Similarly, a standard curve was generated using anti-human anti-SSB/La antibody (LS-C8426; Lifespan Biosciences™, USA) spiked in a Blocker Casein solution (Thermo Fisher Scientific™, USA).

For the anti-SSB assay, the whole saliva samples were diluted in Blocker Casein at a volume ratio of 1:4. For both assays 30 μl of diluted saliva were loaded onto each electrode coated with capture antigen and then incubated for 30 min. Following incubation each electrode was washed for 3 cycles in a PBS and 10% Tween 20 buffer.

Detector Antibody

The secondary antibody, biotinylated polyclonal IgG (H+L) (Thermo Fisher Scientific™, USA) was diluted in Blocker Casein to a concentration of 2500 ng/mL for the anti-SSA assay and to a concentration of 2.5 ng/mL for the anti-SSB assay. For both assays 30 μl of the diluted antibody was pipetted onto each electrode and then incubated for 30 min. Following incubation, each electrode was washed for 3 cycles with a buffer of 1×phosphate buffered saline (PBS, Affymetrix, USA) and 0.05% Tween 20 (BioRad, USA).

Reporter

For the final incubation, Pierce™ Streptavidin Poly-horseradish peroxidase (Thermo Fisher Scientific) was diluted in a Blocker Casein solution (1:2000) and 30 μl of the diluted antibody was loaded onto each electrode and incubated for 30 min followed by 3 wash cycles, as described in previous steps.

Readout

Finally, 60 μl of the 3,3′,5,5′-tetramethyl-benzidine (TMB) substrate solution (34028; Life Technologies) was pipetted onto each sensor. The readout was performed by applying a potential of −200 mV for 60 s to each sensor.

Statistical Analysis

The discriminatory performance of anti-SSA/Ro and -SSB/La measured in saliva was assessed using the area under the receiver operating characteristics (ROC) curves. The associated 95% confidence interval was constructed using DeLong's method to estimate the variance. The strength of association between salivary anti-SSA/Ro and -SSB/La and pSS was measured by the odds ratio and associated 95% confidence interval. The correlation between serum and saliva measurements of anti-SSA/Ro and -SSB/La was assessed using graphical scatter and box plots and quantified using Spearman's rank correlation. A bootstrap procedure was used to construct the associated 95% percentile confidence interval. Due to the nature of this study being explorative and not hypothesis testing, confidence intervals are presented rather than p-values, and no sensitivity and specificity tests were performed. The ROC curves were constructed by estimating the sensitivity and specificity associated with all possible thresholds for the anti-SSA/Ro and anti-SSB antibodies, respectively. The area under the ROC curves and odds ratio are thus the most appropriate measures to quantify the discriminatory ability of the biomarkers.

The experimental results are now described.

EFIRM assays were developed for salivary detection of anti-SSA/Ro and anti-SSB/La. Using a cohort of SS (33) and SICCA (36) patients, EFIRM detected anti-SSA/Ro and anti-SSB/La in saliva of all (100%) SS patients. Unexpectedly and remarkedly is the EFIRM detected anti-SSA/Ro and anti-SSB/La in 100% of SICCA subjects.

EFIRM Immuno-Assays Development for Detection of Anti-SSA/Ro and Anti-SSB/La Antibodies in Saliva

Salivary anti-SSA/Ro and anti-SSB/La have been discovered to be candidate biomarkers for distinguishing SS from SICCA subjects. Efforts to validate these autoantibodies in saliva of SS and SICCA subjects have been challenged by the inability of commercially available bead-based Luminex (Life Technologies/Thermo Fisher Scientific, Carlsbad Calif.) and ELISA-based assays, including clinical laboratories (Zeus Scientific, Branchburg N.J.) that are performing CLIA-assays for serum anti-SSA and anti-SSB detection, to quantitatively and qualitatively detect anti-SSA/Ro and anti-SSB/La in saliva.

An electrochemical assay, electric field-induced released and measurement (EFIRM) for detection of salivary anti-SSA/Ro and anti-SSB/La was developed. The EFIRM assay has superb analytical and clinical performance in the detection of omics targets (ctDNA and proteomic) in liquid biopsy assays for circulating tumor DNA (ctDNA).

The EFIRM immuno-assays for saliva anti-SSA/Ro and anti-SSB/La detection were developed by functionalizing human SSA/Ro and SSB/La onto the gold surface of EFIRM electrodes (FIG. 1). Salivary anti-SSA/Ro and anti-SSB/La will bind to the gold surface-anchored SSA or SSB where a secondary horseradish peroxidase conjugated reporter anti-Ig antibody can then bind to the patient's auti-SSA/Ro or anti-SSB/La. In the presence of the MTB substrate, 20,000 electron transfer will occur per binding as an electrical readout. The process is recyclable in the presence of H₂O₂ as a redox amplification reaction. The EFIRM immuno-assays was further optimized using concentrations of human anti-SSA/Ro and SSB/La to generate optimal calibration curves by spiking authentic patients' anti-SSA/Ro or anti-SSB/La into unstimulated whole saliva samples from healthy donors to demonstrate that the targets were titratable as well as demonstrating low background in non-SS, non-SICCA control samples. Results are shown in FIG. 2.

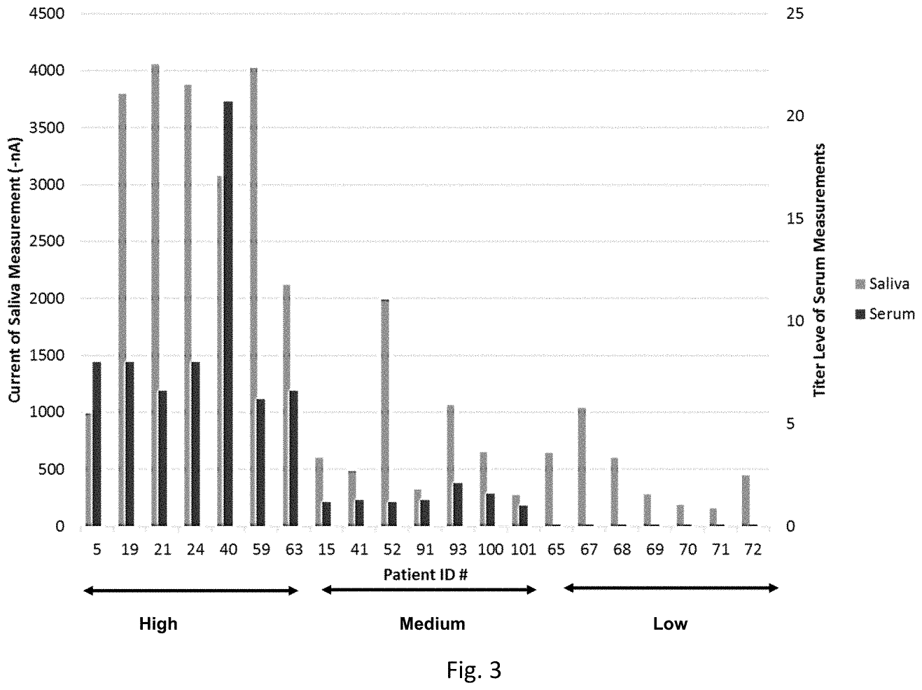

As serum anti-SSA/Ro and anti-SSB/La are current clinical serological criteria for Sjögren's classification (American College of Rheumatology), it is important to demonstrate if the EFIRM measurable anti-SSA/Ro and anti-SSB/La in saliva bears correlation/concordant to serum levels. To test this important clinical attribute, a cohort of SS (33) and SICCA (36) subjects from the Department of Rheumatology at Seoul National University Hospital [SNUH]) were used, where paired serum and saliva samples were collected. From the 33 SS subjects, 21 were randomly selected based on serum levels of anti-SSA/Ro (clinically assayed at SNUH: 7 pSS with low (≤1), medium (1-3) and high (6-21) anti-SSA/Ro-titer in serum). Patients saliva volumes were only sufficient to permit anti-SSA/Ro evaluation. EFIRM assay for salivary anti-SSA/Ro of these 21 saliva samples, blinded and randomized, were performed at UCLA. The analysis of serum anti-SSA/Ro with saliva anti-SSA/Ro revealed a significantly correlation (r=0.75, p=0.0001) (FIG. 3), supporting that EFIRM measurements of salivary anti-SSA/Ro and anti-SSB/La are concordance with serum levels.

Detection of Anti-SSA/Ro and Anti-SSB/La Antibodies in Saliva and Serum of SS and SICCA Subjects

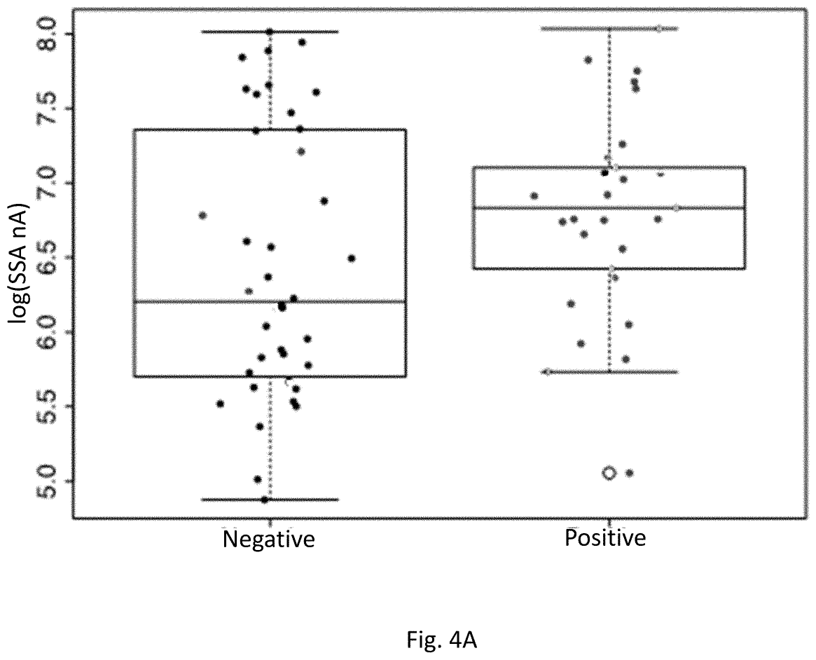

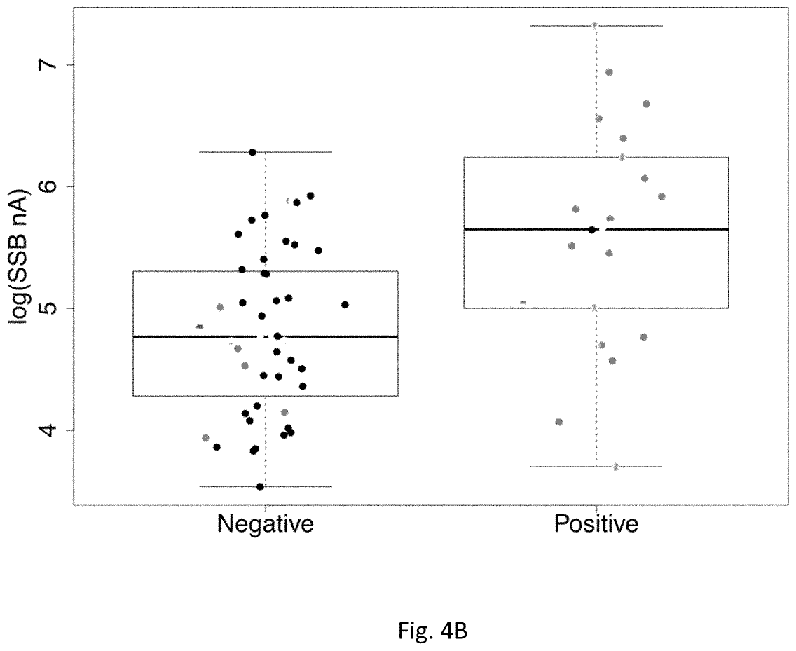

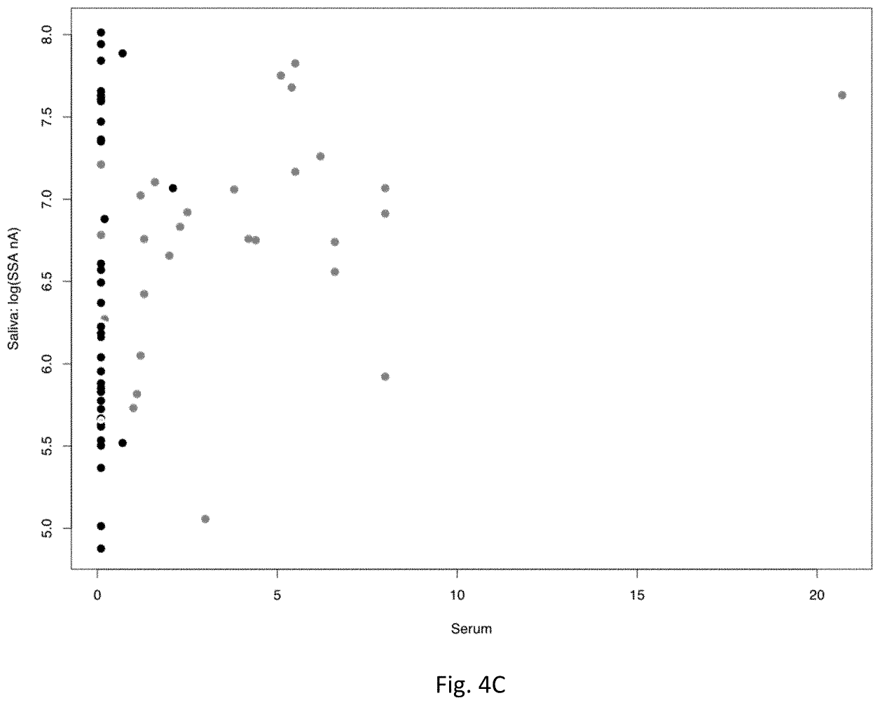

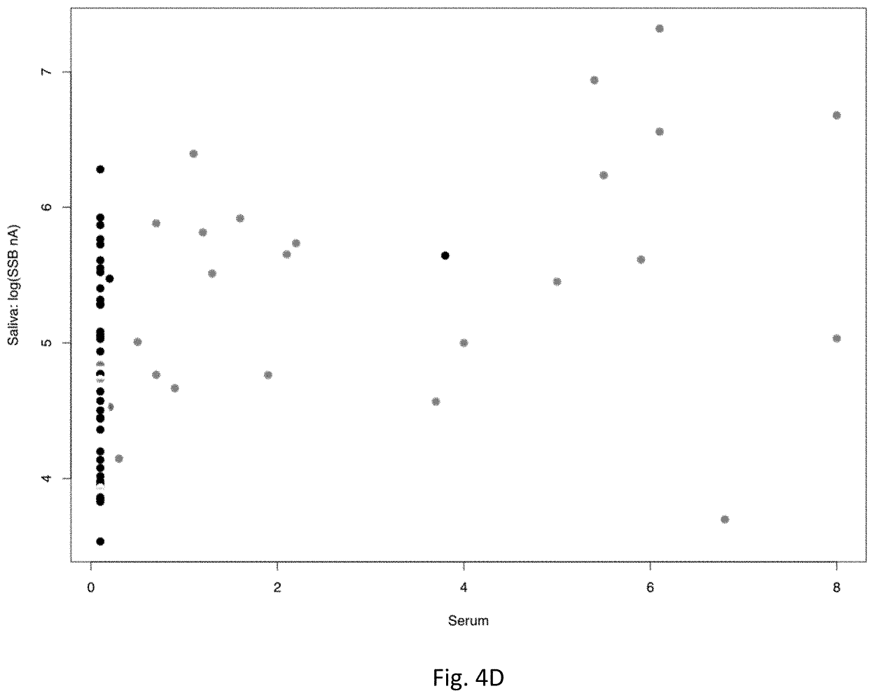

EFIRM's ability to quantitatively measure anti-SSA/Ro and anti-SSB/La in saliva of SS subjects allows for examination of the distribution of these two Sjögren's syndrome pathopneumonic autoantibodies in paired serum and saliva samples of SS and SICCA subjects. FIG. 4A and FIG. 4B, showed the EFIRM measurements (log scale) of salivary levels of anti-SSA/Ro (FIG. 4A) and anti-SSB/La (FIG. 4B), plotting the 33 SS and 36 SICCA subjects according to ACR classification: clinically classified SS and negative for SICCA. FIG. 4A showed that all serum anti-SSA/Ro positive SS subjects (28/33, 85%) have measurable anti-SSA/Ro levels in saliva (Positive column, range ˜5-8 log[SSA nA]). Of importance is that the five serum anti-SSA/Ro negative pSS subjects (5/33, 15%, range ˜5.6-7.2 log[SSA nA]), have readily measurable saliva anti-SSA/Ro activities (Negative column). Collectively EFIRM detected anti-SSA/Ro in saliva of all (100%) SS patients. Surprisingly, EFIRM readily detected salivary anti-SSA/Ro in all serum negative SICCA subjects (35/35, 100%, range ˜4.8-8.0 log[SSA nA])). One clinically classified SICCA subject that is serum positive for anti-SSA/Ro is also saliva positive for anti-SSA/Ro by EFIRM. Collectively all SICCA subjects (100%) have EFIRM detectable anti-SSA/Ro levels in saliva. The range of EFIMR detectable anti-SSA/Ro in saliva is similar between SS (5 to 8 log[SSA nA]) and SICCA (4.8 to 8.0 log[SSA nA]).

A similar behavior and profile of EFIRM detection of saliva anti-SSB/La was observed. FIG. 4B showed that all serum anti-SSB/La positive SS subjects (24/36, 67%) have measurable anti-SSB/La levels in saliva (Positive column, ˜3.6 to 7.2 log[SSB nA]). Of the 12 serum anti-SSB/La negative SS subjects (12/36, 33%), EFIRM detected anti-SSB/La activities in all these 12 SS subjects (Negative column, range ˜4.1 to 5.8 log[SSB nA]). EFIRM detected anti-SSB/La in saliva of all (100%) SS patients. For the SICCA subjects, similar to anti-SSA/Ro detection, 35/35 (100%) of SICCA subjects that are serum negative for anti-SSB/La, are all positive for anti-SSB/La in saliva. One SICCA subject that is serum positive for anti-SSB/La also has EFIRM detectable saliva anti-SSB/La (˜5.5 log[SSB nA]). Collectively all SICCA subjects (100%) have EFIRM detectable anti-SSB/La levels.

The finding that EFIRM detected anti-SSA/Ro and anti-SSB/La in saliva of all SS patients (serum positive and negative) and all serum negative SICCA subjects prompted the examination of the distribution of serum vs saliva anti-SSA/Ro and anti-SSB/La. FIG. 4C and FIG. 4D showed the serum vs saliva distribution of anti-SSA/Ro and anti-SSB/La respectively. 28/33 (85%) and 25/36 (67%) of the SS patients have detectable serum anti-SSA/Ro (FIG. 4C) and anti-SSB/La (FIG. 4D), respectively. 5/33 (15%) and 12/69 (33%) of the SS patients have no detectable serum anti-SSA/Ro or anti-SSB/La activity. All have detectable saliva anti-SSA/Ro and anti-SSB (100%).

The ubiquitous presences of anti-SSA/Ro and anti-SSB/La in saliva of SS and SICCA subjects suggested the utility of these saliva auto-antibody biomarkers for screening and/or risk assessment of SS and SICCA from healthy non-SS, non-SICCA subjects. To test this hypothesis, saliva from 41 non-SS, non-SICCA matched controls were procured. Saliva from these 41 control subjects together with the original 69 SS and sicca patients were independently EFIRM assay for anti-SSA/Ro (FIG. 5). The volume of saliva from the original 69 SS/sicca cohort were not sufficient to permit an independent EFRIM assay for anti-SSB/La.

FIG. 5A shows the data plot of saliva anti-SSA/Ro distribution in SS, SICCA and healthy control subjects. The p values of the differences between the three groups are significant for SS vs Controls, SICCA vs Controls, and [SS+SICCA] vs Controls, but not significant between SS vs SICCA. The area under the ROC curves (AUC) to determine the performance of the salivary anti-SSA/Ro to differentiate SS vs Control is 0.963 (FIG. 5B); SICCA vs Controls is 0.862 (FIG. 5C) and [SS+SICCA] vs Controls is 0.909 (FIG. 5D).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A device for detecting at least one autoantibody in a subject, comprising:
an array of units on a substrate, each unit comprising an electrode chip including a working electrode, a counter electrode, and a reference electrode;
wherein the working electrode of at least one unit is coated with a conducting polymer embedded or functionalized with at least one capture antigen, wherein at least one capture antigen is a target antigen of an autoantibody.

2. The device of claim 1, wherein the target antigen is selected from the group consisting of:
a) the 52 kDa SSA subunit (Ro52) or a fragment thereof,
b) the 60 kDa SSA subunit (Ro60) or a fragment thereof, and
c) the SSB La antigen or a fragment thereof.

3. A method of detecting at least one autoantibody in a subject comprising:
obtaining a saliva sample from the subject;
adding a first portion of the sample mixture to an electrode chip on a device of claim 1;
contacting the sample with a secondary antibody, wherein the secondary antibody is linked to a detectable moiety for generating a current; and
measuring the current in the electrode chip, wherein a change in current is correlated to the presence of at least one autoantibody in the sample.

4. The method of claim 3, wherein the target antigen is selected from the group consisting of:
a) the 52 kDa SSA subunit (Ro52) or a fragment thereof;
b) the 60 kDa SSA subunit (Ro60) or a fragment thereof, and
c) the SSB La antigen or a fragment thereof.

5. The method of claim 4, wherein the autoantibody is selected from the group consisting of an anti-SSA/Ro autoantibody and an anti-SSB/La autoantibody.

6. A method of diagnosing a subject as having or being at increased risk of an autoimmune disease, the method comprising:
obtaining a saliva sample from the subject;
adding a first portion of the sample mixture to an electrode chip on a device of claim 1;
contacting the sample with a secondary antibody, wherein the secondary antibody is linked to a detectable moiety for generating a current; and
measuring the current in the electrode chip, wherein a change in current is correlated to the presence of at least one autoantibody in the sample, wherein the presence of at least one autoantibody is associated with having or being at increased risk of an autoimmune disease.

7. The method of claim 6, wherein the target antigen is selected from the group consisting of:
a) the 52 kDa SSA subunit (Ro52) or a fragment thereof;
b) the 60 kDa SSA subunit (Ro60) or a fragment thereof, and
c) the SSB La antigen or a fragment thereof.

8. The method of claim 7, wherein the autoantibody is selected from the group consisting of an anti-SSA/Ro autoantibody and an anti-SSB/La autoantibody.

9. The method of claim 8, wherein the disease or disorder is selected from the group consisting of Sjogren's Syndrome, Sicca syndrome, rheumatoid arthritis, multiple sclerosis, type I diabetes, systemic lupus erythematosus (SLE), “antinuclear antibody (ANA)-negative” SLE, neonatal lupus erythematosus, idiopathic inflammatory myopathies (IIM), mixed connective tissue disease (MCTD), and primary biliary cholangitis (PBC).
